(19) United States
(12) Patent Application Publication
(10) Pub. No.: US 2009/0270931 A1
(43) Pub. Date: Oct. 29, 2009

(54) METHOD, MOBILE TERMINAL, AND COMPUTER PROGRAM PRODUCT FOR FACILITATING RESUSCITATION

(75) Inventor: Mikael Liden, Vejbystrand (SE)

Correspondence Address: VOLPE AND KOENIG, P.C., UNITED PLAZA, SUITE 1600, 30 SOUTH 17TH STREET, PHILADELPHIA, PA, 19103, US

(73) Assignee: MOBIVERUS IT SOLUTIONS AB, Vejbystrand (SE)

(21) Appl. No.: 12/429,616

(22) Filed: Apr. 24, 2009

Related U.S. Application Data
(60) Provisional application No. 61/047,543, filed on Apr. 24, 2008.

(51) Int. Cl. A61N 1/39

(52) U.S. Cl. 607/5

(57) ABSTRACT

A method for facilitating resuscitation comprising starting an automatic resuscitation sequence, wherein the automatic resuscitation sequence comprises at least one from the group of: emitting audible instructions, displaying text instructions, displaying images, displaying animations, emitting metronome signals, emitting inflation sounds, displaying a clock showing elapsed time since starting the automatic resuscitation sequence, wherein the displaying of animations is associated with the metronome signals, wherein the displaying of animations is associated with the emitting of blowing sounds.

### TECHNICAL FIELD

The present invention relates to a method, a mobile terminal, and a computer program product for facilitating resuscitation and a method for simulating resuscitation.

### BACKGROUND

Sudden cardiac arrest (SCA) is a leading cause of death in Europe and USA, affecting about 700,000 individuals in Europe a year. SCA is often caused by ventricular fibrillation (VF) wherein the heart loses its coordinated function and stops pumping blood effectively. Victims of cardiac arrest need immediate CPR (cardiopulmonary resuscitation), that is artificial circulation and ventilation. This provides a small but critical blood flow to the heart and brain. Many victims of SCA can survive if bystanders act immediately while VF is still present, but successful resuscitation is unlikely once the rhythm has deteriorated to asystole.

An optimum treatment for VF cardiac arrest is to immediately provide CPR until a defibrillator providing an electrical chock can restart the heart. The immediate CPR also increases the likelihood that a defibrillatory shock will terminate VF and enable the heart to resume an effective rhythm and effective systemic perfusion. Chest compression may be important if a shock cannot be delivered sooner than four or five minutes after collapse. Defibrillation interrupts the uncoordinated depolarization-repolarization process that occurs during VF. If the heart is still viable, its normal pacemakers then resume their function and produce an effective rhythm and resumption of circulation.

A huge problem associated with early bystander CPR is the poor general knowledge of the public. Even if CPR is taught, the newly acquired skills are quickly forgotten if they are not used nor revised. The optimal interval for retraining has not been established, but repeated refresher training at intervals of less than 6 months seems to be needed for most individuals who are not undertaking resuscitation on a regular basis.

There are a variety of methods and devices used for resuscitation training. One type of resuscitation training devices are devices providing audible and/or visual feedback during chest compression. Such a device is for example realized as a glove. These devices are helpful in resuscitation training but do not provide any guidance in real resuscitation situations since people rarely carry these devices with them in the everyday life. Furthermore, these devices only apply to chest compression, not to lung ventilation. Additionally, purchasing these devices constitutes a cost that the general public most likely won't pay.

Another example is a device having several keys providing different audible instructions depending on which emergency key is pressed. This device faces similar problems as the devices disclosed above, that is the device is expensive and the user does probably not have the device with him in real emergency situations.

It is thus a major challenge to spread CPR knowledge and skills to the general public in a cost-efficient way and also to maintain and be able to apply the skills and the knowledge in real resuscitation situations.

The present invention is designed to solve these problems or at least to make these problems less evident.

### SUMMARY

In view of the above, an objective of the invention is to solve or at least reduce the problems discussed above.

Generally, the above objectives are achieved by the attached independent patent claims.

According to a first aspect, the present invention is realized by a method for facilitating resuscitation. The method comprises: starting an automatic resuscitation sequence, wherein the automatic resuscitation sequence comprises at least one from the group of: emitting audible instructions, displaying text instructions, displaying images, displaying animations, emitting metronome signals, emitting blowing sounds, displaying a clock showing elapsed time since starting the automatic resuscitation sequence, wherein the displaying of animations is associated with the metronome signals, wherein the displaying of animations is associated with the emitting of inflation sounds.

This is advantageous since the method provides guidance which facilitates resuscitation. The method provides guidance on which part of the resuscitation sequence to perform when. The method also provides guidance on with what frequency the different parts of the sequence are to be performed and for how long.

The method may comprise generating a reminder of revision of resuscitation knowledge, wherein said generating is performed with a predetermined frequency.

This is advantageous in that the resuscitation knowledge may be continuously updated which results in better resuscitation knowledge.

The method may comprise recording sounds during the automatic resuscitation sequence.

This is advantageous since the recorded sounds may be used to extract information on the resuscitation. This information may be useful to the medical personnel taking over after the resuscitation.

The method may comprise detecting position information and sending the position information to a position information server.

This is advantageous since the position information may be sent to medical personnel that is to continue the resuscitation. The position information may for example be sent automatically when the automatic resuscitation sequence is started or, alternatively, the position information may be sent after the user has confirmed that the position information is to be sent. This is advantageous in that the person performing the resuscitation can focus on performing the resuscitation and does not have to worry about providing the medical personnel with position information.

The method may comprise detecting movement pertaining to chest compression, and providing feedback on the movement pertaining to chest compression.

This is advantageous in that the movement pertaining to chest compression may be adapted to the provided feedback which hence improves the resuscitation.

The method may comprise that the feedback may be at least one from the group of audible and visual.

According to a second aspect, the present invention is realized by a mobile terminal for facilitating resuscitation. The mobile terminal comprises: a display which is arranged to display text instructions, images, and animations relating to an automatic resuscitation sequence, and a and clock which is arranged to display elapsed time since a beginning of the automatic resuscitation sequence, a loudspeaker which is arranged to emit audible instructions, metronome signals, and blowing sounds relating to the automatic resuscitation sequence.

It is to be noted that the second aspect of the invention can be embodied with any combination of features corresponding to any of the features of the first aspect of the invention.

The advantages of the first aspect are equally applicable to the second aspect.

The mobile terminal may be a mobile phone.

The CPR software being arranged on a mobile phone is advantageous in that that SCA arrest can happen anywhere, anytime and people normally carry their mobile phone with them. Another advantage is that the most expensive part of this system is the hardware, which already is invested in by the individual.

The mobile terminal may comprise a microphone which is arranged to record sounds relating to the automatic resuscitation sequence.

The mobile terminal may comprise a movement detector which is arranged to detect movement of the mobile terminal pertaining to chest compression.

The mobile terminal may comprise a reminder generator which is arranged to generate a reminder of revision of resuscitation knowledge, wherein said generating is performed with a predetermined frequency.

The mobile terminal may comprise a device that is arranged for detecting position information and for sending the position information to a position information server.

This is advantageous since the position information may be sent to medical personnel that is to continue the resuscitation. The position information may for example be sent automatically when the automatic resuscitation sequence is started or, alternatively, the position information may be sent after the user has confirmed that the position information is to be sent. This is advantageous in that the person performing the resuscitation can focus on performing the resuscitation and does not have to worry about providing the medical personnel with position information.

According to a third aspect, the present invention is realized by a computer program product, comprising computer program code which is stored on a computer-readable storage medium and which, when executed on a processor, carries out the method according to the first aspect of the invention.

The advantages of the first aspect are equally applicable to the third aspect of the invention.

According to a fourth aspect, the present invention is realized by a method according to the description and the accompanying drawings.

According to a fifth aspect, the present invention is realized by a mobile terminal according to the description and the accompanying drawings.

According to a sixth aspect, the present invention is realized by a method for simulating resuscitation. The method comprises: starting an automatic resuscitation sequence, wherein the automatic resuscitation sequence comprises at least one from the group of: emitting audible instructions, displaying text instructions, displaying images, displaying animations, emitting metronome signals, emitting blowing sounds, detecting movement pertaining to chest compression, displaying a clock showing elapsed time since starting the automatic resuscitation sequence, wherein the displaying of animations is associated with the metronome signals, wherein the displaying of animations is associated with the emitting of inflation sounds.

This is advantageous in that a person wishing to refresh or keep his/her knowledge in resuscitation up to date can practice resuscitation using the method. Furthermore, it is advantageous to practice resuscitation using the method since it is advantageous to have seen and heard the automatic resuscitation sequence when resuscitation is to be performed in a real situation.

The advantages of the first aspect are equally applicable to the third aspect of the invention.

The method may comprise providing feedback on the detected movement pertaining to chest compression.

The method may comprise sending the detected movement pertaining to chest compression to a server, and receiving a result from the server based on a comparison between the detected movement pertaining to chest compression sent to the server and a predetermined value.

This is advantageous in that the method may be realized as for example a competition between different users. The users then feel motivated to play the game and hence keep their resuscitation knowledge up to date.

It is to be noted that the sixth aspect of the invention can be embodied with any combination of features corresponding to any of the features of the first aspect of the invention.

Other objectives, features and advantages of the present invention will appear from the following detailed disclosure, from the attached claims as well as from the drawings.

Generally, all terms used in the claims are to be interpreted according to their ordinary meaning in the technical field, unless explicitly defined otherwise herein. All references to “a/an/the [element, device, component, means, step, etc]” are to be interpreted openly as referring to at least one instance of said element, device, component, means, step, etc., unless explicitly stated otherwise. The steps of any method disclosed herein do not have to be performed in the exact order disclosed, unless explicitly stated.

### BRIEF DESCRIPTION OF THE DRAWINGS

FIG. 1 is a representation of a mobile terminal.

FIG. 2 is a view of a mobile telephone.

FIG. 3 is a representation of sample steps in applying life support.

FIGS. 4-6 show a graphical representation of steps in applying life support.

### DETAILED DESCRIPTION OF AN EMBODIMENT

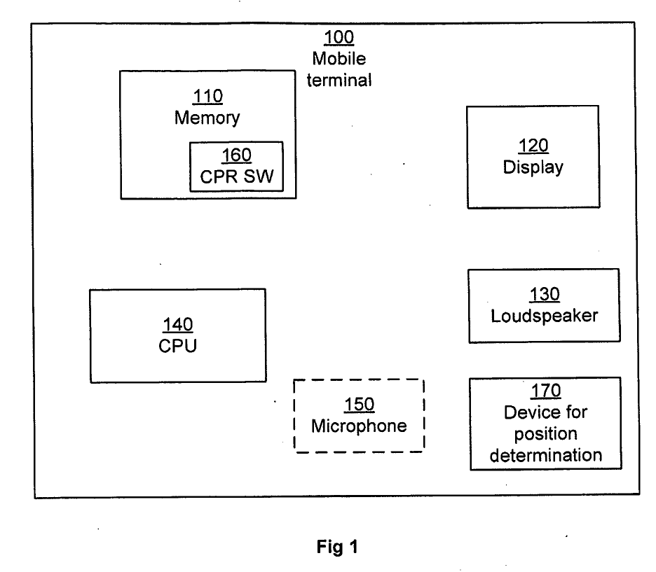

FIG. 1 illustrates a mobile terminal 100, which may for example be a mobile phone, a personal digital assistant (PDA) or the like. The mobile terminal 100 comprises a memory 110, a display 120, a loudspeaker 130, and a CPU (Central Processing Unit) 140. The mobile terminal 100 may also comprise a microphone 150.

A CPR (cardiopulmonary resuscitation) software (SW) 160 for facilitating resuscitation may be stored in the memory 110. The CPR software 160 may be sent to the mobile terminal 100 using for example SMS (Short Message Service), MMS (Multimedia Messaging Service), or an SMS with a hyperlink to a site from where the CPR software 160 may be downloaded. Alternatively, the CPR software 160 may be downloaded from a computer using cable, WiFi, infrared or Bluetooth technology and. Additionally, the mobile terminal 100 may connect to a server and download the CPR software 160.

When executing the CPR software 160, a software application is started which may provide at least one of the following alternatives: an automatic resuscitation sequence, a simulation, a text document, a CPR game, and a short video describing how to perform CPR on a manikin.

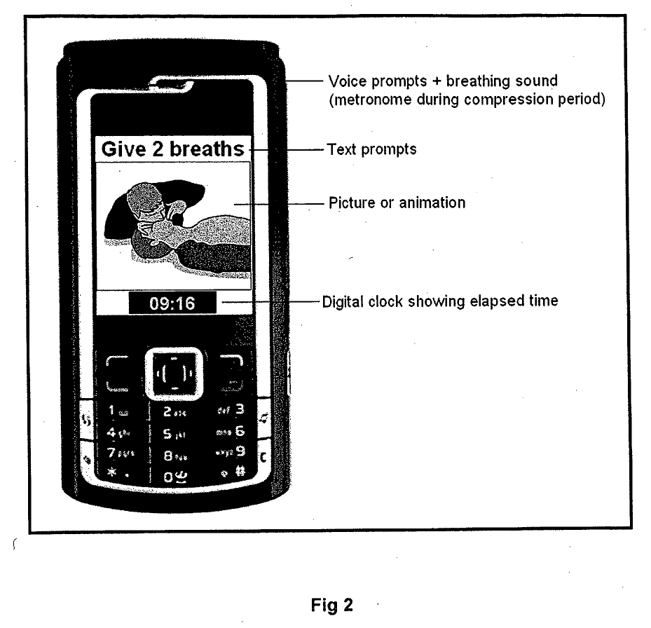

The automatic resuscitation sequence is an automatic sequence providing instructions on how to perform CPR. The automatic resuscitation sequence provides clear, intuitive instructions directly to the user by combining emitting audible instructions, displaying text instructions, displaying images, displaying animations, emitting metronome signals, and emitting blowing sounds, see FIG. 2. The displaying of animations may be synchronized with the emitting of metronome signals and blowing sounds. Furthermore, a clock showing elapsed time since starting the automatic resuscitation sequence is displayed, see FIG. 2. The elapsed time since starting the automatic resuscitation sequence might be useful information for ambulance personnel so that they know for how long time CPR has been performed.

After starting the automatic resuscitation it will, without any interaction by a user, guide the user through the entire resuscitation sequence. During the resuscitation sequence, the microphone 150 may record sounds.

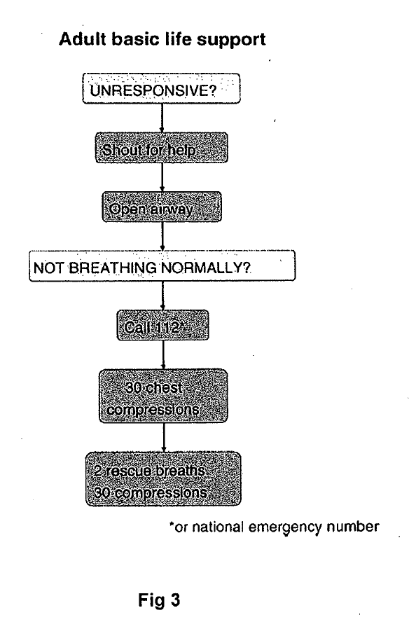

The CPR is to be performed according to methods that have been developed and improved by the European Resuscitation Council (ERC) and the American Heart Association (AHA). These organizations, together with national councils as well as other organizations like the Red Cross continuously work to spread the knowledge and skills of CPR to the general public. See for example FIG. 3.

The CPR software may for example be used for spreading the knowledge of CPR, as a helping tool during a SCA, and for maintaining the CPR knowledge and skills of CPR.

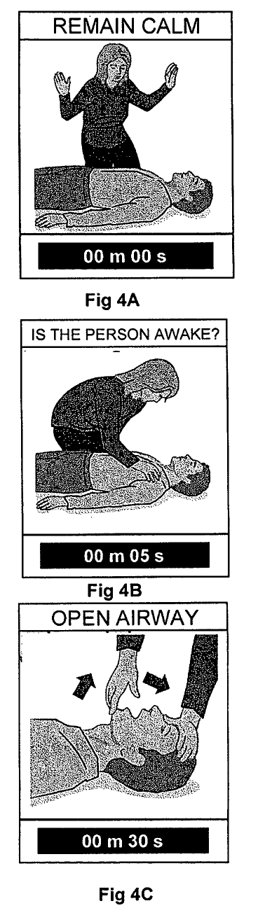

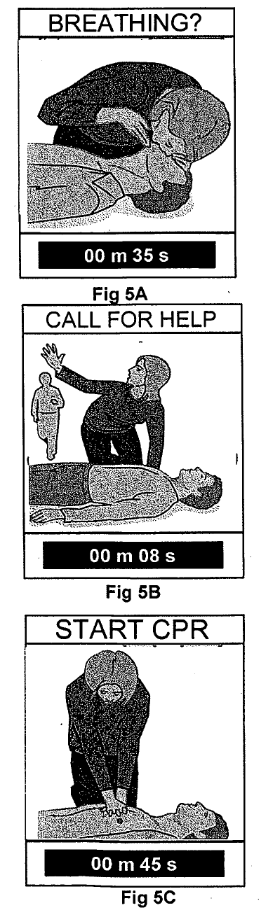

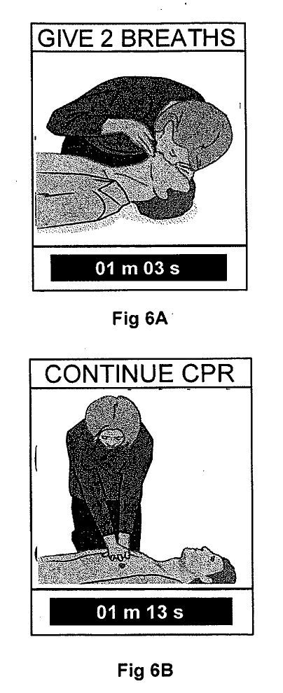

The automatic resuscitation sequence may be realized according to the following description, referring to FIG. 4-6. Text, images and/or animations are displayed on the display 120 according to FIG. 4-6. Audible instructions are emitted according to the following table:

| Figure | |
|---|---|
| no | Audible instructions |
| 4A | Remain calm |
| 4B | Check the persons counciscness by shaking the person |
| | gently and shouting loudly. |
| 4C | Open the persons airway by tilting the head back and lifting |
| | the chin. |
| 5A | Look, listen and feel for signs of breathing. If the person is |
| | breating normally, place the person in recovery position. |
| 5B | If the person is not breathing normally, call the emergency |
| | service number. |
| 5C | Start compressions by placing your hands in the centre of |
| | the persons chest and push firmly 30 times. Follow the |
| | metronome for correct pace and number of compressions. |
| 6A | Give the person two rescue breaths. Change rescuer if |
| | possible. |
| 6B | Continue CPR until professional help arrives or the person |
| | shows signs of life. |

The frequency of the metronome signal may be 100 times per minute. The metronome signal may be emitted 30 times for every sequence of chest compressions. “Give 2 breaths” may be activated after 18 seconds (30 chest compressions). “Continue CPR” may be activated after five seconds. The animation displaying chest compressions may compress 30 times at a rate of 100 per minute.

A reminder may be generated every three months to remind the user of revising his or her CPR knowledge and skills. The revision may for example be performed using the automatic resuscitation sequence, the text document, the CPR game, and/or the short video describing how to perform CPR on a manikin.

Training is to follow the principles of adult education and learning. Generally this will mean an established European Resuscitation Council (ERC) an/or American Heart Association (AHA) course with group participation using interactive discussion and hands-on practice for skills and clinical scenarios for problem-solving and team leadership. The ratio of instructors to candidates should range from 1:3 to 1:6, depending on the type of course.

Core knowledge should be acquired by candidates before the course by study of the course manual or an interactive CD designed for the purpose. The course should aim to produce an improvement in competence in the learner, and there should be a test of core knowledge and an ongoing assessment of practical skills and scenario management. Sophisticated manikins, simulators and virtual reality techniques may be incorporated into the scenario-based training. For basic life support (BLS) by lay people or first responders, home-based learning using a video or interactive CD with a simple manikin may offer a valuable alternative to traditional instructor based courses. This method minimizes candidate disruption and instructor time and finances. However, the role of the instructor should not be underestimated and, in addition to explaining situations that were unforeseen on the original video or CD, the instructor can act as a role model and provide invaluable enthusiasm and motivation. Group participation has also been demonstrated to enhance the overall learning process.

Basic knowledge of CPR may be acquired through the use of Internet, a CD, course literature, or the like.

In one embodiment, the CPR application may use a movement detector arranged on the mobile terminal in order to detect the depth, strength and/or rate of the chest compressions. The mobile terminal is then placed on the chest of the person to be rescued and the rescuer performs the chest compressions on the mobile terminal. The CPR application may then provide feedback to the rescuer on if the performed chest compressions are to be performed differently.

The CPR software being arranged on a mobile phone is advantageous in that that SCA arrest can happen anywhere, anytime and people normally carry their mobile phone with them. Another advantage is that the most expensive part of this system is the hardware, which already is invested in by the individual.

The simulation of the CPR software 160 can be used for training resuscitation. This is advantageous in that a person wishing to refresh or keep his/her knowledge in resuscitation up to date can practice resuscitation using the method. Furthermore, it is advantageous to practice resuscitation using the method since when resuscitation is to be performed in a real situation it advantageous to have seen and heard the automatic resuscitation sequence. Feedback on the detected movement pertaining to chest compression may be provided to the user of the CPR software 160 so that the user is made conscious on how to perform the chest compressions.

The simulation send the detected movement pertaining to chest compression to a server. At the server, results from different users of CPR software 160 may be stored and compared with each other. Results from the comparisons may be sent back to the users. This may be realized as for example a competition between the different users. The users then feel motivated to play the game and hence keep their resuscitation knowledge up to date.

The mobile terminal 100 may comprise a device 170 arranged for position determination. The device 170 may determine a position of the mobile terminal 100. The device 170 may send the position of the mobile terminal 100 to a position determination server.

The invention has mainly been described above with reference to a few embodiments. However, as is readily appreciated by a person skilled in the art, other embodiments than the ones disclosed above are equally possible within the scope of the invention, as defined by the appended patent claims.

1. A method for facilitating resuscitation comprising
starting an automatic resuscitation sequence, wherein the automatic resuscitation sequence comprises at least one from the group of:
emitting audible instructions,
displaying text instructions,
displaying images,
displaying animations,
emitting metronome signals,
emitting inflation sounds,
displaying a clock showing elapsed time since starting the automatic resuscitation sequence,
wherein the displaying of animations is associated with the metronome signals,
wherein the displaying of animations is associated with the emitting of blowing sounds.

2. Method according to claim 1 further comprising generating a reminder of revision of resuscitation knowledge, wherein said generating is performed with a predetermined frequency.

3. Method according to claim 1 further comprising recording sounds during the automatic resuscitation sequence.

4. Method according to claim 1 further comprising detecting position information and sending the position information to a position information server.

5. Method according to claim 1 further comprising
detecting movement pertaining to chest compression, and
providing feedback on the movement pertaining to chest compression.

6. Method according to claim 5, wherein the feedback may be at least one from the group of audible and visual.

7. A mobile terminal for facilitating resuscitation comprising:
a display which is arranged to display text instructions, images, and animations relating to an automatic resuscitation sequence, and a and clock which is arranged to display elapsed time since a beginning of the automatic resuscitation sequence,
a loudspeaker which is arranged to emit audible instructions, metronome signals, and blowing sounds relating to the automatic resuscitation sequence.

8. Mobile terminal according to claim 7, wherein the mobile terminal is a mobile phone.

9. Mobile terminal according to claim 7, further comprising a microphone which is arranged to record sounds relating to the automatic resuscitation sequence.

10. Mobile terminal according to claim 7, further comprising a movement detector which is arranged to detect movement of the mobile terminal pertaining to chest compression.

11. Mobile terminal according to claim 7, further comprising a reminder generator which is arranged to generate a reminder of revision of resuscitation knowledge, wherein said generating is performed with a predetermined frequency.

12. Mobile terminal according to claim 7 further comprising a device that is arranged for detecting position information and for sending the position information to a position information server.

13. A method for simulating resuscitation comprising:
starting an automatic resuscitation sequence, wherein the automatic resuscitation sequence comprises at least one from the group of:
emitting audible instructions,
displaying text instructions,
displaying images,
displaying animations,
emitting metronome signals,
emitting inflation sounds,
detecting movement pertaining to chest compression,
displaying a clock showing elapsed time since starting the automatic resuscitation sequence,
wherein the displaying of animations is associated with the metronome signals,
wherein the displaying of animations is associated with the emitting of blowing sounds.

14. Method according to claim 13 further comprising providing feedback on the detected movement pertaining to chest compression.

15. Method according to claim 13 further comprising sending the detected movement pertaining to chest compression to a server, and
receiving a result from the server based on a comparison between the detected movement pertaining to chest compression sent to the server and a predetermined value.
